(19) United States
(12) Patent Application Publication
(10) Pub. No.: US 2015/0272512 A1
(43) Pub. Date: Oct. 1, 2015

(54) CENTRAL BLOOD PRESSURE ESTIMATION METHOD AND DEVICE THEREOF

(71) Applicants: MICROLIFE INTELLECTUAL PROPERTY GMBH, Heebrugg (CH); FARMAR LICENSING CO., LTD., Taichung City (TW)

(72) Inventors: Chen-Huan Chen, Taipei City (TW); Hao-Min Cheng, Taipei City (TW); Shih-Hsien Sung, Taipei City (TW)

(73) Assignees: NATIONAL YANG-MING UNIVERSITY, Taipei (TW); MICROLIFE INTELLECTUAL PROPERTY GMBH, Heerbrugg (CH)

(21) Appl. No.: 14/437,403

(22) PCT Filed: Jan. 18, 2013
(86) PCT No.: PCT/CN2013/070673

§ 371 Date: Apr. 21, 2015

(30) Foreign Application Priority Data
Oct. 22, 2012 (CN) 201210405274.X

(51) Int. Cl. A61B 5/00; A61B 5/022

(52) U.S. Cl. CPC A61B 5/7278; A61B 5/02225

(57) ABSTRACT

The present invention relates to a central blood pressure estimation device (11). The device comprises a cuff (12); a signal record and storage unit (14) capturing a pressure oscillometric waveform from the cuff and storing the waveform; and an operation and analysis unit obtaining a set of values from the waveform, wherein the values includes a pressure value of the late systolic shoulder produced by wave reflections, an end-systolic pressure value, an area value under the waveform during systole, an area value under the waveform during diastole, a pressure value at end-diastole, and a value of heart rate, and respectively substituting the set of values for corresponding control variables in a linear regression equation to obtain a central blood pressure value, wherein the linear regression equation has a central blood pressure as a dependent variable and has a pressure of the late systolic shoulder produced by wave reflections, an end-systolic pressure, an area under the waveform during systole, an area under the waveform during diastole, a pressure at end-diastole, and a heart rate as the control variables.

### CROSS-REFERENCE TO RELATED APPLICATION

This application is a national stage entry under 35 U.S.C. §371 of International Patent Application No. PCT/CN2013/070673, filed Jan. 18, 2013, which claims priority to China Patent Application No. 201210405274.X filed on Oct. 22, 2012, all of which are incorporated in their entireties by reference herein.

### BACKGROUND OF THE INVENTION

1. Field of the Invention

The invention relates to a central blood pressure estimation method and a device thereof, and, further particularly, to a method and a device for estimating central blood pressures based on a pressure oscillometric waveform from a cuff and a linear regression equation.

2. Description of Related Art

Blood pressure diagnosis is generally conducted based on a systolic blood pressure (SBP) and a diastolic blood pressure (DBP) at brachial arteries. The values of blood pressures (including systolic blood pressure, diastolic blood pressure, etc.) at brachial arteries are commonly measured using a traditional mercury or electronic sphygmomanometer. However, a lot of studies and researches indicated that the systolic blood pressure (SBP-C) read from the central aorta can be preferably used to properly predict cardiovascular events more than the systolic blood pressure read from the brachial arteries can do.

For example, abnormal phenomena, including enhancement of reflected wave, acceleration of pulse conduction and decrease of compliance, frequently occur in the blood flow dynamics of central aorta for hypertension patients. It has been clinically proven that the central blood pressure is a key factor of symptom diagnosis for hypertension patients. The brachial artery blood pressure is determined by measuring the peripheral artery blood pressure trough a mercury or electronic sphygmomanometer, and is usually higher than the central blood pressure, such as the ascending aortic and carotid artery blood pressures. In other words, if a systolic blood pressure can be accurately obtained from central aortas, it is apparently useful to predict the occurrences of hypertension and related cardiovascular events.

US 20090149763 is to disclose a method for remotely estimating central blood pressures. The method proposed a linear regression equation for estimating a systolic blood pressure at ascending aortas. The systolic blood pressure, last phase systolic blood pressure, the sum of the area under the waveform during systole and the area under the waveform during diastole divided by the area under the waveform during diastole, and the pressure of reflected wave hiding beneath the waveform are obtained based on the pulse volume recording waveform from a cuff. The linear regression equation expresses that the foregoing pressures and values serve as four independent variables to determine a dependent variable (a systolic blood pressure at ascending aortas). However, the estimated systolic blood pressure at ascending aortas still has not been acceptable because the statistical data show unwantedly low agreement and broad error scattering (as shown in FIGS. 4 and 5 of the prior art).

To resolve the current technical problems above, the present application puts forth a central blood pressure estimation method and a user-friendly device using the method to accurately estimate central aorta pressures.

### SUMMARY OF THE INVENTION

The present application provides a central blood pressure estimation method and a device thereof. The estimating method can designate critical control variables (independent variables) and give the optimal number of the variables, and well expresses important relation between pulse volume recording waveform and actual (measured) central blood pressure. To estimate central blood pressure is quite accurate by calculating the linear regression equation with the independent variables. Therefore, the estimation method is able to be widely applied to the commercial electric sphygmomanometers.

The present invention provides a central blood pressure estimation device. The device comprises a cuff; a signal record and storage unit capturing a pressure oscillometric waveform from the cuff and storing the waveform; and an operation and analysis unit obtaining a set of values from the waveform, wherein the values includes a pressure value of the late systolic shoulder produced by wave reflections, an end-systolic pressure value, an area value under the waveform during systole, an area value under the waveform during diastole, a pressure value at end-diastole, and a value of heart rate, and respectively substituting the set of values for corresponding control variables in a linear regression equation to obtain a central blood pressure value, wherein the linear regression equation has a central blood pressure as a dependent variable and has a pressure of the late systolic shoulder produced by wave reflections, an end-systolic pressure, an area under the waveform during systole, an area under the waveform during diastole, a pressure at end-diastole, and a heart rate as the control variables. The pressure oscillometric waveform includes a pulse volume recording waveform.

In an embodiment of the present application, the central blood pressure estimation device further comprises an adjustable pressure unit for controlling the pressure in the cuff to be increased, maintained, or decreased. The pulse volume recording waveform is a pressure signal, and is obtained when the adjustable pressure unit constantly maintains the pressure in the cuff.

In an embodiment of the present invention, the central blood pressure is a systolic blood pressure (SBP-C), and the linear regression equation is illustrated below:

SBP-C=s1×SBP2+s2×ESP+s3×As+s4×Ad+a5×DBP+s6×Heart Rate+c1

wherein SBP-C represents a systolic blood pressure, SBP2 represents a pressure value of the late systolic shoulder produced by wave reflections, ESP represents an end-systolic pressure value, As represents an area under the waveform during systole, Ad represents an area under the waveform during diastole, DBP represents a pressure value at end-diastole, and Heart Rate represents a heart rate; s1-s6 and c1 are constants.

In the foregoing embodiment, the constant s1-s6 and c1 are respectively 0.30, 0.20, 1.97, 0.87, −0.75, 1.00 and −58.16.

In an embodiment of the present application, the central blood pressure is a pulse pressure (PP-C), and the linear regression equation is illustrated below:

PP-C=p1×SBP2+p2×ESP+p3×As+p4×Ad+p5×DBP+p6×Heart Rate+c2

wherein PP-C represents a pulse pressure, SBP2 represents a pressure value of the late systolic shoulder produced by wave reflections, ESP represents an end-systolic pressure value, As represents an area under the waveform during systole, Ad represents an area under the waveform during diastole, DBP represents a pressure value at end-diastole, and Heart Rate represents a heart rate; p1-p6 and c2 are constants.

In the foregoing embodiment, the constant p1-p6 and c2 are respectively 0.26, −0.06, 2.61, 1.37, −1.73, 1.62 and −114.64.

The present invention further provides a central blood pressure estimation method. The method comprises: establishing a linear regression equation, wherein the linear regression equation has a pressure of the late systolic shoulder produced by wave reflections, an end-systolic pressure, an area value under the waveform during systole, an area value under the waveform during diastole, a pressure at end-diastole, and a heart rate as the control variables; obtaining a set of values by capturing a pressure oscillometric waveform from the cuff, wherein the values includes a pressure value of the late systolic shoulder produced by wave reflections, an end-systolic pressure value, an area under the waveform during systole, an area under the waveform during diastole, a pressure value at end-diastole, and a value of heart rate; and respectively substituting the set of values for corresponding control variables in the linear regression equation to obtain a central blood pressure value.

### BRIEF DESCRIPTION OF THE DRAWINGS

In order to sufficiently understand the essence, advantages and the preferred embodiments of the present invention, the following detailed description will be more clearly understood by referring to the accompanying drawings.

FIG. 1 is a function block diagram of a central blood pressure estimation device in accordance with the present invention;

FIG. 2 is a schematic diagram of a pressure oscillometric waveform with specified parameters in accordance with the present invention;

FIG. 3 is a flow chart of a central blood pressure estimation method in accordance with the present invention;

FIGS. 4 and 5 are statistical diagrams of Bland-Altman analyses based on the estimated values calculated from the linear regression equation (1) in accordance with the present invention; and

FIGS. 6 and 7 are statistical diagrams of Bland-Altman analyses based on the estimated values calculated from the linear regression equation (2) in accordance with the present invention.

### DETAILED DESCRIPTION OF THE INVENTION

The present application is described below by referring to the embodiments and the figures for a person skilled in the art to easily understand the purpose, technical content, characteristics, and achieved results of the present invention.

The pressure oscillometric waveform is recorded during the blood pressure measurement through an electronic sphygmomanometer. A very accurate central blood pressure (e.g. systolic blood pressure, diastolic blood pressure, the difference (or named pulse pressure; PP) between the systolic blood pressure and diastolic blood pressure, etc.) can be obtained by a linear regression equation so as to adequately predict the occurrences of hypertension and cardiovascular events through diagnosis.

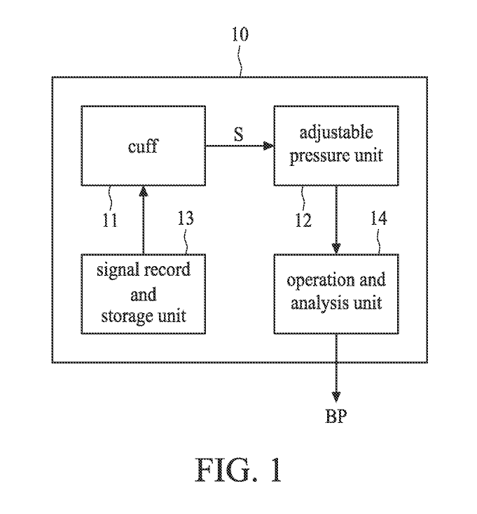

FIG. 1 is a function block diagram of a central blood pressure estimation device in accordance with the present invention. The central blood pressure estimation device 10 comprises a cuff 11, a signal record and storage unit 12, an adjustable pressure unit 13, and an operation and analysis unit 14. The signal record and storage unit 12 and operation and analysis unit 14 can be integrated into a single IC chip component. In other embodiment, the record and storage unit 12 and operation and analysis unit 14 also can be respectively performed by the sub-functions of various IC chip components. Thus, the present invention is not intended to be limited to the embodiment and its figure. A person skilled in the art may know the storage function of the signal record and storage unit 12 can be replaced by a memory.

The cuff 11 is used to firmly wrap around the upper arm. A pressure oscillometric waveform S can be obtained from the inner of the cuff 11. In the embodiment, the pressure oscillometric waveform includes the pulse volume recording waveform.

The signal record and storage unit 12 derives and stores the pressure oscillometric waveform S.

The adjustable pressure unit 13 can increase, maintain or decrease the pressure of the cuff 11 by control. It is worthily noted that the adjustable pressure unit 13 can maintain the air pressure of the cuff 11 as a constant during a certain period. In the embodiment, the adjustable pressure unit 13 can maintain the air pressure within the cuff 11 at constant 60 mmHg for 30 seconds but the present invention is not limited to this. A person skilled in the art may know that the air pressure of the cuff can be adjusted from 40 to 70 mmHg.

The operation and analysis unit 14 can obtain a set of values from the pressure oscillometric waveform. The set of values includes a pressure value of the late systolic shoulder produced by wave reflections (SBP2; or the second peak of the systolic blood pressure), an end-systolic pressure value (ESP), an area value under the waveform during systole (As), an area value under the waveform during diastole (Ad), a pressure value at end-diastole (DBP), and a heart rate. Furthermore, the operation and analysis unit 14 respectively substitutes the set of values for corresponding control variables in a linear regression equation to obtain a central blood pressure value. The areas and points representative of the set of values in the pressure oscillometric waveform will be further described below. Moreover, the establishment and expression of the linear regression equation will be described below.

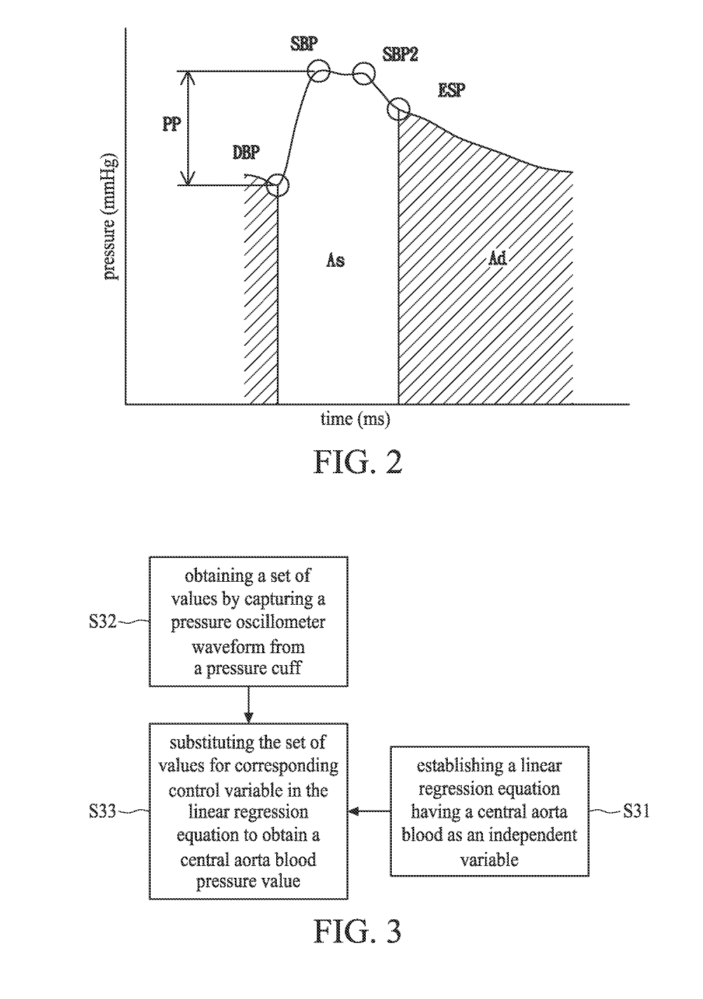

FIG. 2 is a schematic diagram of a pressure oscillometric waveform with specified parameters in accordance with the present invention. The first highest pressure value shown in the pressure oscillometric waveform is a systolic blood pressure (SBP). Within the systolic period, the second highest pressure value or the second peak value is produced by wave reflections as shown in the pressure oscillometric waveform, and is also named a pressure value of the late systolic shoulder produced by wave reflections (SBP2). The pressure value at the end of the systolic period is an end-systolic pressure value (ESP). An area value under the waveform during systole is represented by As. An area value under the waveform during diastole (a hatched period excluding the systolic period) is represented by Ad. The lowest pressure value shown in the pressure oscillometric waveform is a pressure value at end-diastole (DBP).

The linear regression equation has a central blood pressure as a dependent variable and has a pressure of the late systolic shoulder produced by wave reflections, an end-systolic pressure, an area under the waveform during systole, an area under the waveform during diastole, a pressure at end-diastole, and a heart rate as the control variables.

the linear regression equation is illustrated below:

SBP-C=0.30×SBP2+0.20×ESP+1.97×As+0.87×Ad−0.75×DBP+1.00×Heart Rate−58.16 (1)

PP-C=0.26×SBP2−0.06×ESP+2.61×As+1.37×Ad−1.73×DBP+1.62×Heart Rate−114.64 (2)

In the equations (1) and (2), SBP-C represents a systolic blood pressure and PP-C represents a pulse pressure. The regression coefficient (being constant) before each control variable and constants (−58.16, −114.64) are just exemplary. The coefficients and constants can be varied according to various estimation devices or electronic components used in the devices, but the present invention is not limited to the example.

FIG. 3 is a flow chart of a central blood pressure estimation method in accordance with the present invention. The method can be applied to the central blood pressure estimation device 10, and also can be applied to common electronic sphygmomanometers for improving their functions. In Step S31, the blood pressure signals are obtained by invasively and noninvasively measuring the blood pressure of subjects so that the actual central blood pressures and brachial artery blood pressure of the subjects can be obtained. A linear regression equation is established by the multivariate analysis of variance. Some specified parameters are derived from the pressure oscillometric waveform to serve as control variables of the equation (as discussed above) for accurately estimating the central blood pressure. The present invention designates six control variables which have very strong relationship with the central blood pressure so that the central blood pressure can be exactly estimated. The present invention is not limited to the control variables.

In Step 32, the cuff 11 of the central blood pressure estimation device 10 is firmly wrapped around the upper arm of a subject. A set of values is obtained by capturing a pressure oscillometric waveform from the cuff 11. The values includes a pressure value of the second peak SBP2 produced by wave reflections during systole, an end-systolic pressure value ESP, an area As under the waveform during systole, an area Ad under the waveform during diastole, a pressure value at end-diastole DBP, and a heart rate (Heart Rate). The pressure oscillometric waveform analysis includes dynamic oscillometric waveform analysis (recorded during the decreasing period of pressure in the cuff) and static oscillometric waveform analysis (recorded when the pressure in the cuff decreased to a constant pressure, i.e. pulse volume recording waveform).

The air pressure of the cuff wrapped around the upper arm may be maintained at constant 60 mmHg when a common electronic sphygmomanometer is used to measure the brachial artery blood pressure (including a systolic blood pressure, an average pressure, a diastolic blood pressure and a heart rate). In the meanwhile, the blood passing through the brachial arteries causes the skin surface of the upper arm increased to act against the pressure from the cuff. Simultaneously, the volume of the cuff is accordingly changed. When the volume of the cuff is decreased, the pressure of the cuff varies and the variation is recorded as PVR waveform. Generally speaking, the PVR waveform has very strong relationship with the actual brachial artery blood pressure waveform or the actual central blood pressure. However, some specified points located on the PVR waveform may be changed because a different cuff with various characteristics is used. Therefore, the accuracy of the estimated central blood pressure is accordingly affected. The present invention can improve the accuracy of the estimated pressure through the above steps and following steps.

Afterward, the set of values obtained in Step S32 from the pressure oscillometric waveform are substituted for the corresponding control variables in the linear regression equation to obtain a central blood pressure value, as shown in Step 33.

In the embodiment, the central blood pressure may be a systolic blood pressure SBP-C and a pulse pressure PP-C. A person skilled in the art may know that the estimated pressure can be the difference between the systolic blood pressure and diastolic blood pressure, an average pressure, a diastolic blood pressure or related pressure referable to clinical diagnosis.

In view of above, the foregoing linear regression equation may be applied to common electronic sphygmomanometers capable of recording PVR waveform signals, and a central blood pressure can be estimated or predicted based on the PVR waveform signals. The current related technical filed even requires various instruments which only can be operated by a professional person to adequately estimate the central blood pressure so that it is inconvenient for the users of common electronic sphygmomanometers. The present invention can resolve the inconvenient problem and improve the accuracy of the estimated central blood pressure. Therefore, the central blood pressure estimation technique of the present invention can be widely applied to home care service and clinical diagnosis.

The foregoing linear regression equation is established as a central blood pressure estimation model by the multivariate analysis of variance based on the sufficient blood pressure samples which are obtained by invasively and noninvasively measuring the blood pressure of subjects. The detailed description is as follows:

### Linear Regression Equation Establishment

An arterial catheter is used for the direct and invasive measurement, and is inserted into the central aorta of subjects of the first group for recording the central aorta pressure waveform. In the embodiment, the catheter is sent to an ascending aorta. It comprises a Siemens-approved transducers with a resistance of 200-3000 Ohms and an equivalent pressure sensitivity of 5 μV/V/mmHg±10%. At the meanwhile, the left arm of the subject is wrapped by a cuff for recording the PVR signals in the cuff at constant 60 mmHg during a certain period (e.g. 10 seconds). A mean waveform can be obtained by averaging several PVR signal waveforms respectively within various heart beat periods during the certain period.

The central aorta pressure waveforms and the pressure oscillometric waveforms in the cuff measured from the subjects of the first group can be used to establish the linear regression equations (1) and (2) by the multivariate analysis of variance for estimating the central aorta pressure. In the embodiment, the mean pressure oscillometric waveform is calibrated to the systolic blood pressure and diastolic blood pressure. Afterward, some control variables (or parameters) are obtained based on the calibrated waveforms. The present invention can evaluate the effects of the control variables so as to find out the most important six control variables by which the central aorta pressure and pulse pressure as the independent variables of the linear regression equations are respectively expressed. The control variables can improve the accuracy of the estimated central aorta pressure. The number of them is optimal so that the calculation cost is quite saved.

### Linear Regression Equation Verification

The data are obtained by invasively and noninvasively measuring the subjects of the second group, and are used to verify the linear regression equations (1) and (2). As a result, the estimated values derived from the linear regression equations (1) and (2) are quite accurate. The accuracy of the estimated values can meet the requirements suggested by the European Society of Hypertension International Protocol. The established and verified results are shown in Table 1 below.

TABLE 1
Measured and Estimated Results

| Characteristics | First Group | Second Group |
|---|---|---|
| and pressures | (56 subjects) | (85 subjects) |
| of subjects | Mean ± SD | Mean ± SD |
| Man, % | 66.1 | 69.4 |
| Age (years) | 65.5 ± 13.7 | 64.8 ± 13.6 |
| Height (cm) | 162.4 ± 10.5 | 163.8 ± 7.8 |
| Weight (Kg) | 68.8 ± 13.1 | 68.1 ± 11.7 |

Actual (Measured) invasive measurement result (mmHg)

| | | |
|---|---|---|
| Aortic SBP | 141 ± 27 | 135 ± 22 |
| Aortic DBP | 68 ± 12 | 70 ± 12 |
| Aortic PP | 73 ± 26 | 64 ± 23 |

Actual (Measured) noninvasive measurement result (mmHg)

| | | |
|---|---|---|
| Brachial SBP | 138 ± 23 | 132 ± 18 |
| Brachial DBP | 76 ± 11 | 76 ± 10 |
| Brachial PP | 62 ± 20 | 56 ± 16 |
| Baseline heart beat | 69 ± 10 | 69 ± 12 |
| (beats/min) | | |

Estimated result (mmHg)

| | | |
|---|---|---|
| Aortic SBP | 141 ± 25 | 134 ± 20 |
| Aortic DBP | 69 ± 13 | 70 ± 10 |
| Aortic PP | 73 ± 25 | 64 ± 21 |

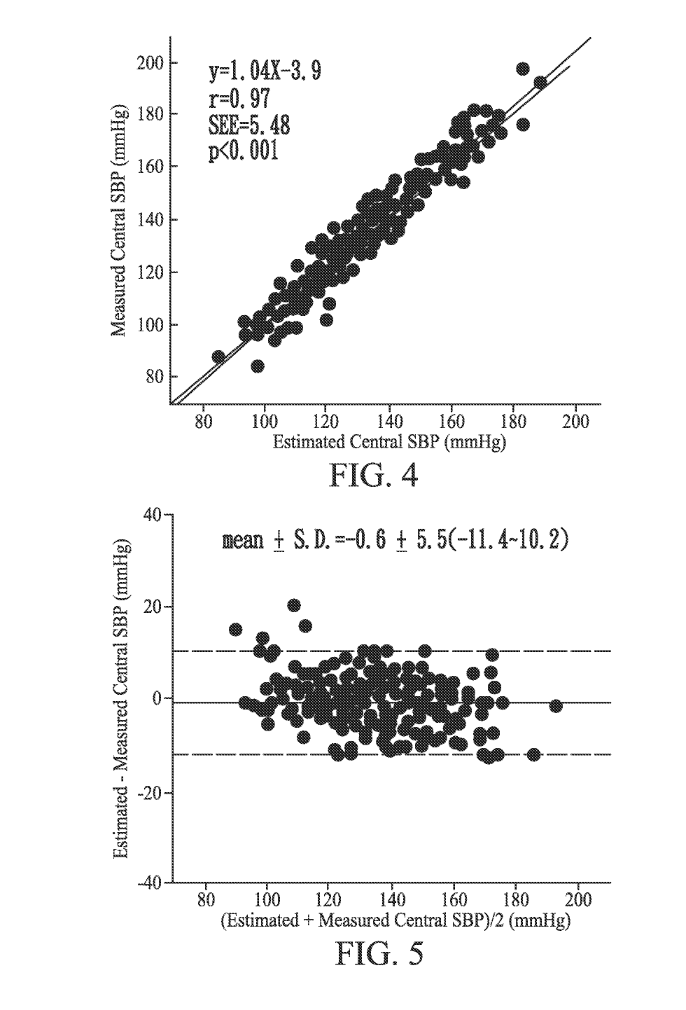

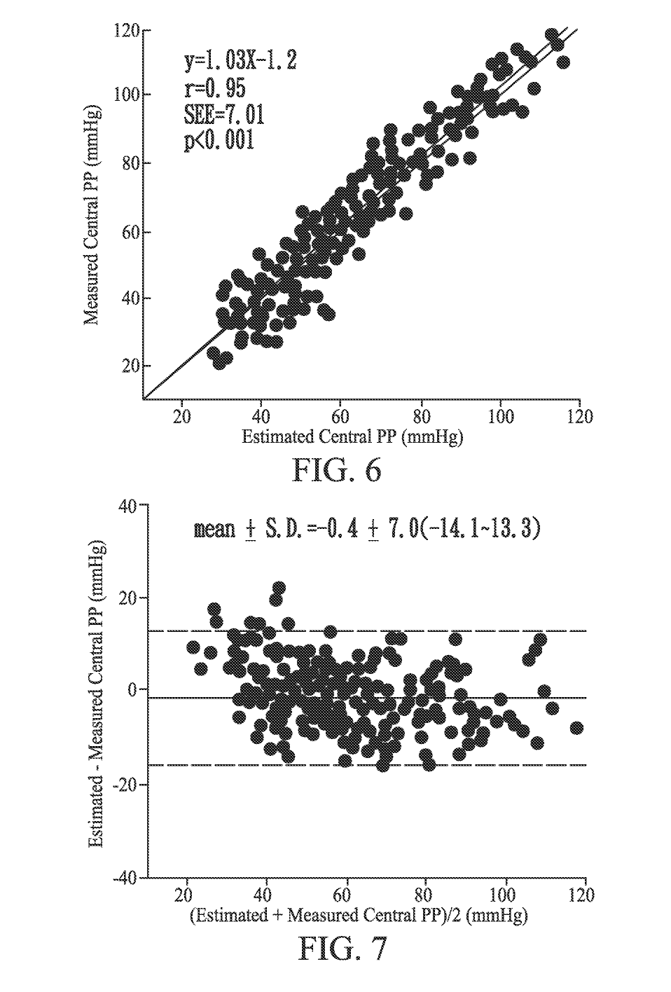

In order to further verify the estimated results derived from the linear regression equations (1) and (2) by various statistical indexes, further central aorta pressure waveforms and the pressure oscillometric waveforms in the cuff are obtained from the other set of 225 subjects for performing Bland-Altman Analyses. FIGS. 4 and 5 are statistical diagrams of Bland-Altman analyses based on the estimated values calculated from the linear regression equation (1). FIGS. 6 and 7 are statistical diagrams of Bland-Altman analyses based on the estimated values calculated from the linear regression equation (2).

FIG. 4 shows excellent agreement between the estimated and measured central aortic SBP and very high relationship between them. FIG. 5 is an error statistical diagram illustrating the difference subtracting the measured central aortic SBP from the estimated central aortic SBP. Most of errors are scattered within two standard deviations (SD), and no remarkable systematic drift was observed.

FIG. 6 shows excellent agreement between the estimated and measured central aortic PP and very high relationship between them. FIG. 7 is an error statistical diagram illustrating the difference subtracting the measured central aortic PP from the estimated central aortic SBP. Most of errors are scattered within two standard deviations (SD), and no remarkable systematic drift was observed. Furthermore, the estimated central aortic DBP can be obtained by subtracting DBP calculated by the linear regression equation (1) from PP calculated by the linear regression equation (2).

The foregoing embodiments of the present invention have been presented for the purpose of illustration. Although the invention has been described by certain preceding examples, it is not to be construed as being limited by them. They are not intended to be exhaustive, or to limit the scope of the invention. Modifications, improvements and variations within the scope of the invention are possible in light of this disclosure. For example, the processing or calibration of waveform signals can be changed in sequence. Moreover, another function block can be added to or inserted into the function block diagram of the central blood pressure estimation device 10, but the added function block such as a filter and a screen showing estimated values may not affect the technical concept of the present invention.

What is claimed is:

1. A central blood pressure estimation device, comprising:
a cuff;
a signal record and storage unit capturing a pressure oscillometric waveform from the cuff and storing the pressure oscillometric waveform; and
an operation and analysis unit obtaining a set of values from the pressure oscillometric waveform, the values including a pressure value of the late systolic shoulder produced by wave reflections, an end-systolic pressure value, an area value under the waveform during systole, an area value under the waveform during diastole, a pressure value at end-diastole, and a heart rate;
wherein the operation and analysis unit respectively substitutes the set of values for corresponding control variables in a linear regression equation to obtain a central blood pressure value.

2. The central blood pressure estimation device according to claim 1, further comprising:
an adjustable pressure unit controlling an air pressure in the cuff to be increased, maintained, or decreased.

3. The central blood pressure estimation device according to claim 2, wherein the pressure oscillometric waveform comprises a pulse volume recording waveform.

4. The central blood pressure estimation device according to claim 3, wherein the pulse volume recording waveform is a pressure signal, and the pressure signal is obtained when the adjustable pressure unit constantly maintains the air pressure in the cuff.

5. The central blood pressure estimation device according to claim 1, wherein the central blood pressure is a systolic blood pressure (SBP-C), and the linear regression equation is illustrated below:
SBP-C=s1×SBP2+s2×ESP+s3×As+s4×Ad+a5×DBP+s6×Heart Rate+c1
wherein SBP-C represents a systolic blood pressure, SBP2 represents a pressure value of the late systolic shoulder produced by wave reflections, ESP represents an end-systolic pressure value, As represents an area under the waveform during systole, Ad represents an area under the waveform during diastole, DBP represents a pressure value at end-diastole, and Heart Rate represents a heart rate; s1-s6 and c1 are constants.

6. The central blood pressure estimation device according to claim 5, wherein the constant s1-s6 and c1 are respectively 0.30, 0.20, 1.97, 0.87, −0.75, 1.00 and −58.16.

7. The central blood pressure estimation device according to claim 1, wherein the central blood pressure is a pulse pressure (PP-C), and the linear regression equation is illustrated below:
PP-C=p1×SBP2+p2×ESP+p3×As+p4×Ad+p5×DBP+p6×Heart Rate+c2
wherein PP-C represents a pulse pressure, SBP2 represents a pressure value of the late systolic shoulder produced by wave reflections, ESP represents an end-systolic pressure value, As represents an area under the waveform during systole, Ad represents an area under the waveform during diastole, DBP represents a pressure value at end-diastole, and Heart Rate represents a heart rate; p1-p6 and c2 are constants.

8. The central blood pressure estimation device according to claim 7, wherein the constant p1-p6 and c2 are respectively 0.26, −0.06, 2.61, 1.37, −1.73, 1.62 and −114.64.

9. A central blood pressure estimation method, comprising:
establishing a linear regression equation, wherein the linear regression equation has a plurality of control variables;
obtaining a set of values by capturing a pressure oscillometric waveform from a cuff, wherein the values includes a pressure value of the late systolic shoulder produced by wave reflections, an end-systolic pressure value, an area under the waveform during systole, an area under the waveform during diastole, a pressure value at end-diastole, and a heart rate; and
respectively substituting the set of values for corresponding control variables in the linear regression equation to obtain a central blood pressure value.

10. The central blood pressure estimation method according to claim 9, wherein the pressure oscillometric waveform comprises a pulse volume recording waveform.

11. The central blood pressure estimation method according to claim 10, wherein the pulse volume recording waveform is a pressure signal, and the pressure signal is obtained when the air pressure in the cuff is constantly maintained.

12. The central blood pressure estimation method according to claim 9, wherein the central blood pressure is a systolic blood pressure (SBP-C), and the linear regression equation is illustrated below:
SBP-C=s1×SBP2+s2×ESP+s3×As+s4×Ad+a5×DBP+s6×Heart Rate+c1
wherein SBP-C represents a systolic blood pressure, SBP2 represents a pressure value of the late systolic shoulder produced by wave reflections, ESP represents an end-systolic pressure value, As represents an area under the waveform during systole, Ad represents an area under the waveform during diastole, DBP represents a pressure value at end-diastole, and Heart Rate represents a heart rate; s1-s6 and c1 are constants.

13. The central blood pressure estimation method according to claim 12, wherein the constant s1-s6 and c1 are respectively 0.30, 0.20, 1.97, 0.87, −0.75, 1.00 and −58.16.

14. The central blood pressure estimation method according to claim 9, wherein the central blood pressure is a pulse pressure (PP-C), and the linear regression equation is illustrated below:
PP-C=p1×SBP2+p2×ESP+p3×As+p4×Ad+p5×DBP+p6×Heart Rate+c2
wherein PP-C represents a pulse pressure, SBP2 represents a pressure value of the late systolic shoulder produced by wave reflections, ESP represents an end-systolic pressure value, As represents an area under the waveform during systole, Ad represents an area under the waveform during diastole, DBP represents a pressure value at end-diastole, and Heart Rate represents a heart rate; p1-p6 and c2 are constants.

15. The central blood pressure estimation method according to claim 14, wherein the constant p1-p6 and c2 are respectively 0.26, −0.06, 2.61, 1.37, −1.73, 1.62 and −114.64.
